(19) United States
(12) Patent Application Publication
(10) Pub. No.: US 2021/0396695 A1
(43) Pub. Date: Dec. 23, 2021

(54) APPARATUS AND METHODS FOR THERMALLY TESTING A SAMPLE

(71) Applicant: Oxford University Innovation Limited, Oxford (Oxfordshire) (GB)

(72) Inventors: Kamaljit Singh CHANA, Oxford (Oxfordshire) (GB); Vikram SRIDHAR, Oxford (Oxfordshire) (GB); Saleema KHIMJI, Toronto (CA)

(21) Appl. No.: 17/293,096

(22) PCT Filed: Nov. 15, 2019
(86) PCT No.: PCT/GB2019/053254

§ 371 Date: May 12, 2021

(30) Foreign Application Priority Data
Nov. 20, 2018 (GB) 1818888.8

(51) Int. Cl. G01N 25/18; G01N 33/15

(52) U.S. Cl. CPC G01N 25/18; G01N 33/15

(57) ABSTRACT

The invention provides an apparatus and a method for testing a sample, with a sensor element (4) comprising a first conductor (301), a second conductor (302) and a third conductor (303) in series. The first conductor (301) is connected to the second conductor (302) at a first sensing junction (316A) and the second conductor (302) is connected to the third conductor (303) at a second sensing junction (316B). Heating is applied to a sample via Joule heating in the second conductor (303). A thermal response is measured (12) via a potential difference between the first conductor and the third conductor generated by the Seebeck effect.

The present invention relates to testing samples, particularly pharmaceutical products for the purpose of detecting counterfeit medication in a low cost and/or portable device.

The World Health Organisation (WHO) currently estimates that 1 in 10 medical products in low- and middle-income countries is substandard or falsified. In a 2017 WHO study involving more than 48,000 medicines, 65% of fake medicines were found to involve drugs for treating malaria and bacterial infections. Every year, more than 122,000 African children under the age of five lose their lives as a result of counterfeit antimalarials alone. Both generic and innovator medicines can be falsified, ranging from very expensive products for cancer to very inexpensive products for treatment of pain.

Currently a suspected drug would have its package analysed visually to see whether it is a fake. Counterfeit manufacturers are making that job much more difficult. Alternative procedures may use one or more of the following: chemical analysis; chromatography; spectroscopy; chemical induced colour-based testing; hardness and dissolutions testing; infrared & near infrared testing; Raman Spectroscopy; ultraviolet testing; x-ray diffraction (XRD); and x-ray fluorescence (XRF). These alternative procedures are generally costly and/or time consuming and may not be practical at the point of sale, particularly in low- and middle-income countries.

It is an object of the invention to at least partially address one or more of the above problems.

According to an aspect, there is provided an apparatus for testing a sample, comprising: a sensor element comprising a first conductor, a second conductor and a third conductor, wherein: the first conductor, second conductor and third conductor are connected together electrically in series; the first conductor is connected to the second conductor at a first sensing junction; the second conductor is connected to the third conductor at a second sensing junction; and the apparatus further comprises a measurement unit configured to: apply heating to a sample under test via Joule heating in the second conductor, the Joule heating being generated by driving an electrical current in series through the first conductor, second conductor and third conductor; measure a thermal response of the sample to the heating by measuring a potential difference between the first conductor and the third conductor, wherein the potential difference is influenced by a temperature gradient along the first conductor and a temperature gradient along the third conductor via the Seebeck effect; and generate an indication of whether the sample satisfies a predetermined criterion based on the measured thermal response.

Thus, an apparatus is provided that can be manufactured using reliable and rugged components at low cost. Components that have been mass produced for traditional thermocouple devices may be used to provide the first and third conductors and some of the electronics used to measure temperature in the region of the first and second sensing junctions. In comparison with a traditional thermocouple device, the second conductor effectively provides an extended junction region between components (the first and third conductors) that perform a role similar to the positive and negative legs of the thermocouple device. However, the second conductor provides the additional possibility of applying heating directly in the location where the thermal response is to be measured without requiring separate electrical connections and a separate resistive heating element. Errors associated with differences in location between the heating and the measurement of the thermal response are avoided. The averaging effect on the measurement of temperature provided by the extended junction region further reduces errors and reduces the risk of anomalies due to local variations or spikes in temperature. The robustness of the sensor element, in comparison with alternative approaches based on thin film measurements for example, also means that the heating can be applied for a relatively long period of time. This allows compositional properties to be sampled to a greater depth in the case where the sample (e.g. pharmaceutical product) is solid, which may be used to reduce errors by further spatial averaging or to detect anomalies beneath the surface of the sample.

The measurement unit can be implemented based on electronics for obtaining a temperature measurement from a standard thermocouple, which is widely available and low cost. Adaptation to provide the heating capability can also be implemented at low cost. Combining the above advantages with the relatively low power requirements means that the apparatus can easily be implemented in portable form, for example in a hand-held device. The apparatus is thus suitable for use in the field, such as in pharmacies, shops, or health facilities in remote locations, and even in a domestic context.

In an embodiment, the electrical resistance of the second conductor is at least 2 times higher than the electrical resistance of the first conductor and than the electrical resistance of the third conductor. The Joule heating thus occurs predominantly in the second conductor. This minimizes overall power requirements and helps to focus heating to the region being tested, which improves accuracy.

In an embodiment, the second conductor is locally elongate and has a length that is at least 2 times longer than a shortest distance between the first sensing junction and the second sensing junction. Thus, high resistance can be achieved without a cross-sectional area of the second conductor needing to become excessively small (which might reduce ruggedness and/or increase cost of manufacture by requiring manufacturing steps having higher levels of precision). Additionally, the spatial separation between the first and second sensing junctions can be kept relatively small, which ensures that the measurements based on the Seebeck effect accurately reflect the thermal response of the region between the first and second sensing junctions.

In an embodiment, at least part of the first conductor, second conductor, and third conductor are embedded in a matrix material; and one or more of the following are flush with an outer surface of the matrix material and can be brought into direct contact with the sample under test: the first sensing junction, the second sensing junction, the second conductive material. This arrangement can be manufactured efficiently by embedding the components in the material matrix and machining (e.g. lapping) the matrix material until the components are made flush with the outer surface. The components flush with the outer surface can be brought into good thermal contact with the sample to be tested, thereby providing good sensitivity, while at the same time being maximally supported by the matrix material, which enhances robustness, longevity and reliability.

According to an alternative aspect, there is provided a method of testing a sample, comprising: providing a sensor element comprising a first conductor, a second conductor and a third conductor, wherein: the first conductor, second conductor and third conductor are connected together electrically in series; the first conductor is connected to the second conductor at a first sensing junction; the second conductor is connected to the third conductor at a second sensing junction; and the method further comprises: applying heating to a sample under test via Joule heating in the second conductor, the Joule heating being generated by driving an electrical current in series through the first conductor, second conductor and third conductor; measuring a thermal response of the sample to the heating by measuring a potential difference between the first conductor and the third conductor, wherein the potential difference is influenced by a temperature gradient along the first conductor and a temperature gradient along the third conductor via the Seebeck effect; and generating an indication of whether the sample satisfies a predetermined criterion based on the measured thermal response.

Embodiments of the invention will now be described, by way of example only, with reference to the accompanying drawings in which corresponding reference symbols indicate corresponding parts, and in which:

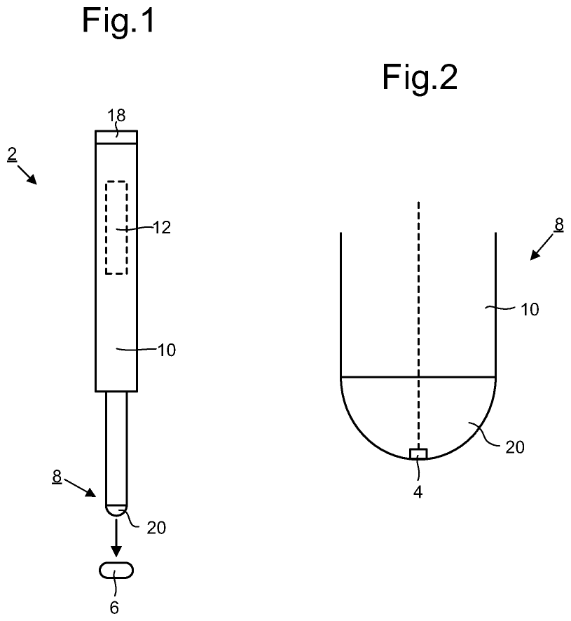

FIG. 1 schematically depicts an example apparatus for testing a pharmaceutical product as sample;

FIG. 2 is a magnified view of a probe tip of the apparatus of claim 1;

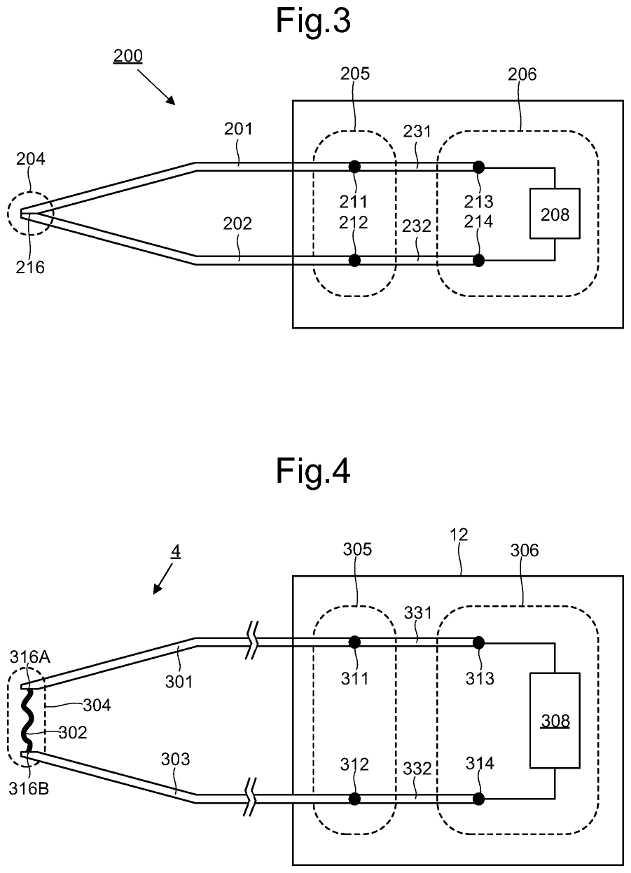

FIG. 3 schematically depicts a thermocouple device;

FIG. 4 schematically depicts a sensor element of the apparatus of FIG. 1;

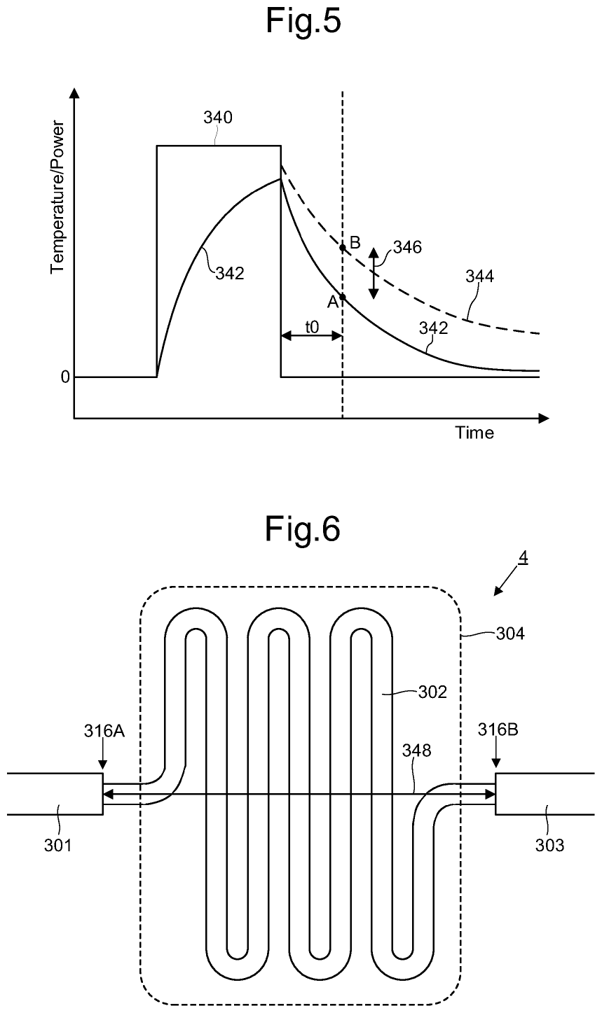

FIG. 5 is a graph schematically depicting application of a heating pulse and a variation in temperature with time measured by the sensor element;

FIG. 6 is a schematic end view of a sensor element showing a serpentine shaped second conductor;

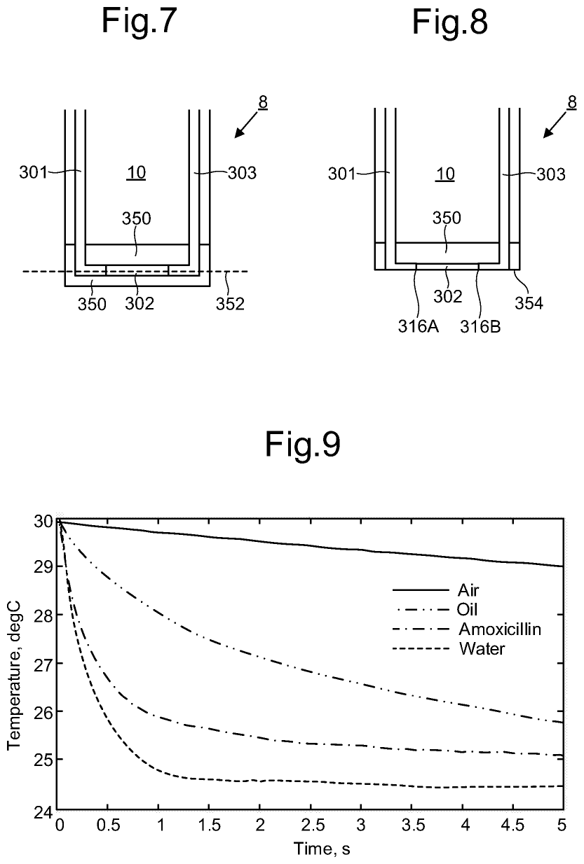

FIG. 7 is a schematic side sectional view of a probe tip before lapping away of a matrix material during manufacture;

FIG. 8 is a schematic side sectional view of the probe tip of FIG. 7 after lapping away of the matrix material to make first and second sensing junctions flush with an outer surface of the matrix material;

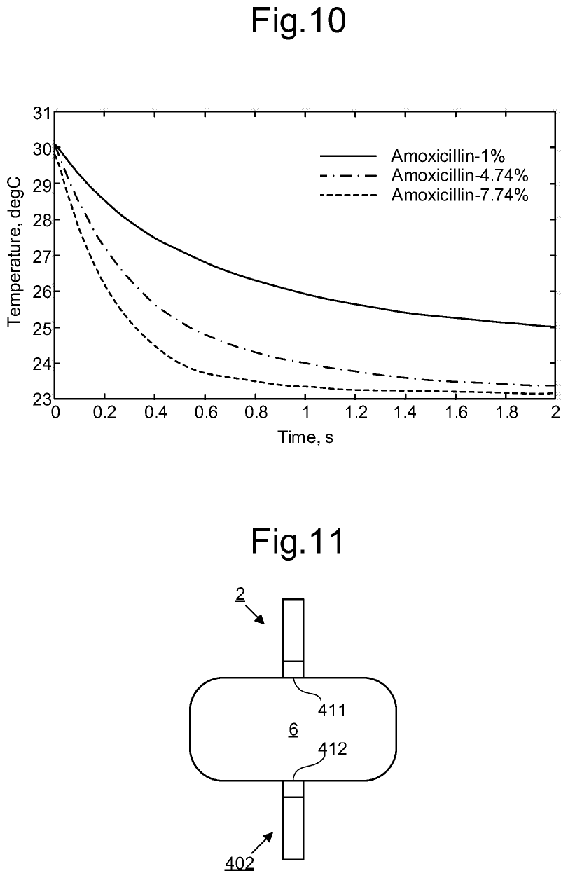

FIGS. 9 and 10 are graphs depicting experimental results using an apparatus of the type depicted in FIG. 1 for different materials; and

FIG. 11 depicts testing of a sample via sensor elements engaging with opposite sides of the sample.

Embodiments of the present disclosure provide apparatus and methods for testing a sample, for example a pharmaceutical product, to determine whether the sample satisfies a predetermined criterion. The predetermined criterion may relate to whether a pharmaceutical product is a genuine version of the pharmaceutical product, whether the pharmaceutical product has reached a predetermined level of quality or purity, and/or whether the pharmaceutical product contains less than a desired minimum amount of a target substance or more than a desired maximum amount of an undesired impurity. The testing procedure may thus comprise determining compositional information about a pharmaceutical product. The compositional information may comprise any compositional property that affects thermal characteristics, in particular heat transfer characteristics, of the pharmaceutical product. The compositional information may comprise chemical or structural information. The sample may comprise a solid, for example in the form of a tablet or powder. In an embodiment, the sample comprises a solid body for oral administration (e.g. a tablet), the solid body comprising a pharmaceutically active ingredient. In other embodiments, the sample comprises a liquid, such as a medicine in liquid form.

The methods use a sensor element to apply heating to the sample (e.g. pharmaceutical product). A thermal response (which may be referred to also as a temperature response) of the sample is measured. The thermal response is dependent on a heat transfer characteristic of the sample. The heat transfer characteristic depends on chemical and/or structural properties of the sample. The measured thermal response therefore provides information about chemical and/or structural properties of the sample. The heat transfer characteristic affects how efficiently heat will be conducted away from the sensor element. Applied heat penetrates underneath the surface of the sample being sensed, allowing sub-surface structure to be sensed, such as different layers of a pharmaceutical product, or inhomogeneities or inclusions within a pharmaceutical product. The methodology is thus sensitive to pharmaceutical products in which an active or other component is distributed within a matrix material having a different composition. The ability to detect sub-structure makes it possible for the sensing to be performed through materials separating the sensor element from the material of interest, including not only outer layers of a pharmaceutical product but also packaging or other materials that may be present around the pharmaceutical product.

Sensing can be achieved effectively even for relatively low heating power. The method can be performed for example without increasing the local temperature of the sample by more than a few degrees Celsius (e.g. 2-5 degrees Celsius). Thermal damage to the sample is therefore avoided. Overall power requirements are also kept low, facilitating implementation as a portable (e.g. battery powered) device.

Heat transfer characteristics of materials (e.g. thermal properties such as thermal conductivity, κ, specific heat capacity, c, and quantities that depend on one or both of these properties) can depend sensitively on the composition (e.g. chemical or structural) of the materials. The thermal product, \sqrt{ρcκ}, where ρ is equal to the density, is often a heat transfer characteristic that is particularly sensitive to composition because it takes into account both κ and c. Changes in either or both of κ and c will typically result in a change in \sqrt{ρcκ}. Changes in relative concentrations of different components in a multi-component material can be detected where the different components have different thermal properties. Changes in structure can be detected where there is a density or compositional change.

FIGS. 1 and 2 depict an example apparatus 2 for testing a sample 6, such as a pharmaceutical product. The apparatus 2 comprises a sensor element 4 configured to be brought into thermal contact with the sample (e.g. in direct contact with a surface of the sample or in contact via a thin coupling layer or gel not forming an integral part of the sample 6). In the embodiment shown, the sensor element 4 is provided at the distal end of a probe tip 8. The apparatus may comprise a body 10 that a user can grasp to bring the sensor element 4 into thermal contact with the sample 6. The apparatus 2 may have a shape and/or size that is similar to a writing pen, for example. Where the sample 6 is a liquid, the probe tip 8 may be immersed in the liquid to test the liquid.

The apparatus 2 comprises a measurement unit 12. The measurement unit 12 applies heating to the sample 6 via the sensor element 4. The measurement unit 12 measures a thermal response to the heating to determine compositional information about the sample 6. The thermal response is dependent on a heat transfer characteristic of the sample 6, as discussed above. The measurement unit 12 uses the measured thermal response to provide an indication about whether the sample 6 satisfies a predetermined criterion.

In an embodiment, the probe tip 8 comprises a deformable coupling member 20 that deforms on engagement with the sample 6 when the probe tip 8 is brought into contact with the sample 6. The sensor element 4 is mounted on, in, and/or in thermal contact with, the deformable coupling member 20. The deformable coupling member 20 may be configured to deform elastically (e.g. such that the deformable coupling member 20 is resilient and springs back to an equilibrium shape when the contact between the probe 8 and the sample 6 is removed). In an embodiment, the deformable coupling member 20 comprises a foam material or a deformable membrane (pocket) comprising a fluid such as air. An example arrangement is shown in FIG. 2, where the sensor element 4 is provided on or near a distal surface of the deformable coupling member 20. The broken line indicates schematically an electrical connection path from the sensor element 4 towards the measurement unit 12. The electrical connection path may pass through or around the deformable coupling member 20 depending on the nature of the deformable coupling member 20. Where the deformable coupling member 20 comprises a membrane containing a fluid such as air, the electrical connection path may comprise metallic tracks formed on a surface of the membrane.

The deformable coupling member 20 helps a user to maintain a suitable force between the probe 8 and the sample 6 during testing, thereby improving accuracy and repeatability of the measurements.

The principle of operation of the sensor element 4 builds on that of traditional thermocouples, so a short discussion of this is now given with reference to FIG. 3.

FIG. 3 depicts a common measurement configuration for a thermocouple device 200. The thermocouple device 200 comprises a first leg 201 and a second leg 202. The first leg 201 and the second leg 202 are electrically connected together, for example by welding, to form a sensing junction 216. The first leg 201 and the second leg 202 are conductors (e.g. wires) formed from different materials. The first leg 201 is connected at junction 211 to a first measurement wire 231. The second leg 202 is connected at junction 212 to a second measurement wire 232. The first and second measurement wires 231 and 232 are connected at junctions 213 and 214 to a measurement circuit 208. The measurement circuit 208 measures a potential difference between the junctions 213 and 214 in order to determine a temperature Tₛ at the sensing junction 216.

The potential difference indicative of the temperature Tₛ at the sensing junction 216 arises because of the Seebeck effect. In a K-type thermocouple, for example, the first leg 201 is formed from chromel and the second leg 202 is formed from alumel. The first and second measurement wires 231 and 232 are formed from the same material as each other (e.g. copper). The temperature in region 205 (containing junctions 211 and 212) is known and may be denoted T₀. The temperature in region 204 (containing the sensing junction 216) is then obtained as follows.

The Seebeck effect describes the electromotive force that arises when there is a temperature gradient in an electrically conductive material. When there is no internal current flow, the gradient of the voltage, ΔV, is related to the gradient in temperature, ΔT, by the Seebeck coefficient S(T), which is a temperature dependent material property, as follows:

ΔV=−S(T)ΔT

The potential difference between 213 and 214 does not depend on the temperature in region 206 because the first and second measurement wires 231 and 232 are formed from the same material and have the same temperature gradient along them. The potential difference depends entirely on the properties of the first and second legs 201 and 202 and the temperature difference between T₀ and Tₛ. The potential difference is given by the following expression:

∫_{T}_{₀}^{T}^{ₛ}(S₊(T)−S₋(T))dT

where S₊ and S₋ are the Seebeck coefficients of the materials forming the first and second legs 201 and 202 (chromel and alumel in the example above). A characteristic function E(T) for a given thermocouple can be established using calibration measurements and the potential difference V between 213 and 214 will be given by the following expression:

V=E(Tₛ)−E(T₀)

where

E(T)=∫(S₊(T′)−S₋(T′))dT′+constant

FIG. 4 depicts a sensor element 4 and a measurement unit 12 suitable for use with the apparatus of FIG. 1. The sensor element 4 comprises a first conductor 301 (e.g. a wire), a second conductor 302 (e.g. a wire) and a third conductor 303 (e.g. a wire). The first conductor 301, second conductor 302 and third conductor 303 are connected together in series between junctions 313 and 314 connected to measurement circuit 308. The measurement circuit 308 may be implemented to provide at least the functionality provided by the measurement circuit 208 used for the thermocouple device 200 of FIG. 3 (namely, to measure a potential difference between junctions 313 and 314 caused by the Seebeck effect).

The first conductor 301 is connected to the second conductor 302 (e.g. by welding) at a first sensing junction 316A. The second conductor 302 is connected to the third conductor 303 (e.g. by welding) at a second sensing junction 316B.

The measurement unit 12 applies heating to a sample (e.g. pharmaceutical product) under test provided in region 304 via Joule heating in the second conductor 302. The Joule heating is generated by the measurement circuit 308 driving an electrical current through the series of first conductor 301, second conductor 302 and third conductor 303. The electrical current also flows through first and second measurement wires 331 and 332 respectively connecting the first conductor 301 and the third conductor 303 to the measurement circuit 308.

As in the thermocouple device 200 of FIG. 3, the first and second measurement wires 331 and 332 are formed from the same material (e.g. copper). The first measurement wire 331 is connected to the first conductor 301 at junction 311. The second measurement wire 332 is connected to the third conductor 303 at junction 312. Region 305 containing the junctions 311 and 312 is held at a common temperature (i.e. junction 311 is always at the same temperature as junction 312). This may be achieved for example by arranging for the junctions 311 and 312 to be in good thermal contact with each other and/or with a block of material having high thermal conductivity and/or high heat capacity. The region 306 containing junctions 313 and 314 may typically be thermally isolated from the region 305 to avoid heat generated in the measurement circuit 308 perturbing the temperature in the region 305.

The measurement circuit 308 measures a temperature in the region 304 in thermal contact with the second conductor 302 and thereby measures a thermal response of the sample 6 (which is also in thermal contact with the region 304, for example by being in direct contact with the second conductor 302). The measurement of the thermal response can be performed during or after the application of the Joule heating via the second conductor 302. In the example discussed below with reference to FIG. 5, the heating is applied before the measurement of the thermal response. Thus, the heating of the sample 6 and the measurement of the thermal response are performed in non-overlapping time periods.

The measurement of the thermal response is performed by measuring a potential difference between the first conductor 301 and the third conductor 303. The potential difference is influenced by the Seebeck effect associated with a temperature gradient along the first conductor 301 and a temperature gradient along the third conductor 303. In the example of FIG. 4, the temperature gradient along the first conductor 301 is between the temperature in region 305 and the temperature at the first sensing junction 316A. The temperature gradient along the third conductor 303 is between the temperature in region 305 and the temperature at the second sensing junction 316B.

The first conductor 301 consists of a first material. The third conductor 303 consists of a second material. The first material and the second material are different, such that a potential difference generated by the temperature gradient along the first conductor 301 is different to the potential difference generated by the temperature gradient along the third conductor 303. In an embodiment, the first and second materials are selected to match pairs of materials used for the positive and negative legs of traditional thermocouple devices such as the thermocouple device 200 discussed above with reference to FIG. 3. For example, the first material and the third material may respectively comprise, consist essentially of, or consist of one of the following pairs of materials: chromel and constantan as for a type E thermocouple device; iron and constantan as for a type J thermocouple device; chromel and alumel as for a type K thermocouple device; 82% Ni/18% Mo and 99.2% Ni/0.8% Co, by weight, as for a type M thermocouple device; Nicrosil and Nisil as for a type N thermocouple device; copper and constantan as for a type T thermocouple; 70% Pt/30% Rh and 94% Pt/6% Rh, by weight, as for a type B thermocouple device; 87%Pt/13%Rh by weight and platinum as for a type R thermocouple device; 90% Pt/10% Rh by weight and platinum as for a type S thermocouple device; 95% W/5% Re and 74% W/26% Re, by weight, as for a type C thermocouple device; 97% W/3% Re and 75% W/25% Re, by weight, as for a type D thermocouple device; and Tungsten and 74% W/26% Re by weight as for a type G thermocouple device.

In an embodiment, as depicted in FIG. 5, the heating of the sample 6 is performed by driving a constant current through the second conductor 302 for a predetermined period of time. A square wave heating pulse 340 is thus applied to the sample 6 (in thermal contact with region 304).

The curve 342 schematically depicts how the temperature of the sample 6 is expected to change as a function of time. The temperature increases during the heating pulse 340 and decreases outside of the heating pulse 340. The measurement of the temperature by the sensor element 4 is performed outside of the heating pulse 340. This is an example of a class of embodiments in which the heating and the measurement of the thermal response are performed in non-overlapping time periods. Performing the heating and the measurement of the thermal response in non-overlapping time periods simplifies the electronics required to implement the measurement unit 12 significantly.

In an embodiment, a response to the heating pulse is compared with the response to a corresponding heating pulse applied to a reference material. The size of the response, the variation of the response as a function of time, or various other aspects of the response may be considered. Any deviation from the response measured for the reference material may be used to detect a deviation from normality for the sample 6 being tested. The nature of the heating pulses may be selected to achieve optimum sensitivity for the particular type of sample (e.g. pharmaceutical product) being tested. This may involve selecting particular pulse shapes, amplitudes, durations and/or repetition rates, for example.

The measurement unit 12 generates an indication of whether the sample 6 satisfies a predetermined criterion based on the measured thermal response. In an embodiment, this is achieved by comparing the measured thermal response to a corresponding measured response obtained at an earlier time for a reference sample (e.g. a reference pharmaceutical product). In a case where the apparatus 2 is being used to detect counterfeit drugs, for example, the reference pharmaceutical product 6 may consist of a genuine version of the drug in question. In one implementation, the apparatus 2 is provided with a user input unit 18 (e.g. a button), as exemplified schematically in FIG. 1. A user positions the probe tip 8 so that the sensor element 4 is in thermal contact with a reference pharmaceutical product (e.g. a product known to be of acceptable quality and/or authentic). The user provides an input to the apparatus 2 via the user input unit 18 (e.g. by pressing a button) to cause the apparatus 2 to measure the reference pharmaceutical product by applying a heating pulse to the sensor element 4. The thermal response of the reference pharmaceutical product is stored. The user then repositions the probe tip 8 so that the sensor element 4 is in thermal contact with a pharmaceutical product 6 to be tested. The user provides a further input to the apparatus 2 via the user input unit 18 (e.g. by pressing a button) to cause the apparatus 2 to measure the pharmaceutical product 6 to be tested. The measurement unit 12 compares the measured thermal response from the pharmaceutical product 6 to be tested with the measured thermal response from the reference pharmaceutical product.

In the example of FIG. 5, the measurement unit 12 measures a temperature of the sample 6 at a time point corresponding to a predetermined period of time t0 after the end of the heating pulse 340. An expected variation of temperature with time for the reference sample is indicated by curve 344. The measured temperature at point A is then compared with the expected measured temperature at point B on the curve 344 for the reference sample. If the deviation 346 is less than a first threshold value the measurement unit 12 generates an indication to show that the sample 6 has “passed” the test (and is therefore of sufficiently high quality, where the testing is being done as part of a quality control procedure, or is authentic, wherein the testing is being done to detect fake versions of the pharmaceutical product). This may be indicated for example by illuminating a green LED or by providing a suitable indication or text on a display (not shown). If the deviation 346 is more than a second threshold value but less than a third threshold value, the measurement unit 12 generates an indication to show that the sample 6 is “suspect” and that further investigation may be required. This may be indicated for example by illuminating an orange LED or by providing a suitable indication or text on a display. If the deviation 346 is more than the third threshold value the measurement unit 12 generates an indication to show that the sample 6 has “failed” the test (e.g. is deemed to be a counterfeit version of the drug). This may be indicated for example by illuminating a red LED or by providing a suitable indication or text on a display.

In an embodiment, a majority of the electrical resistance in the circuit through which current is driven to provide the Joule heating is contributed by the second conductor 302. In an embodiment, the electrical resistance of the second conductor 302 is at least 2 times, optionally at least 5 times, optionally at least 10 times, higher than the electrical resistance of the first conductor 301 and than the electrical resistance of the third conductor 303. The Joule heating is thus focussed spatially in the region of the second conductor 302. In an embodiment, the separation between the first and second sensing junctions 316A and 316B is less than the length of either of the first and third conductors 301 and 303. Thus, not only is the overall amount of Joule heating occurring in the second conductor 302 higher than in either of the first and third conductors 301 and 303, the heating is concentrated spatially, leading to a majority of the power per unit volume being delivered in the region 304.

In an embodiment, an average cross-sectional area (e.g. averaged longitudinally) of the second conductor 302 is at least 2 times, optionally at least 5 times, optionally at least 10 times, smaller than an average cross-sectional area (e.g. averaged longitudinally) of the first conductor 301 and than an average cross-sectional area (e.g. averaged longitudinally) of the third conductor 303. This approach helps to achieve high resistance without excessively increasing the length of the second conductor 302.

Alternatively or additionally, as depicted in FIG. 6, the second conductor 302 is locally elongate (e.g. like a wire) and has a length that is at least 2 times, optionally at least 5 times, optionally at least 10 times, longer than a shortest distance 348 between the first sensing junction 316A and the second sensing junction 316B. For example, the conductor 302 may adopt a sinuous form, such as a spiral, a helix or a serpentine shape. An example of a serpentine geometry is depicted in FIG. 6.

FIGS. 7 and 8 depict a probe tip 8 at different stages of manufacture of an example apparatus 2 for testing a sample. In FIG. 7 a distal part of the first conductor 301, the second conductor 302, and a distal part of the third conductor 303 are all embedded in a matrix material 350. Various materials may be used for the matrix material. In an embodiment, the matrix material comprises an acrylic. In a subsequent processing step, the probe tip 8 is lapped down to a target plane 352, which exposes the first and second sensing junctions 316A and 316B, as depicted in FIG. 8. The embodiment of FIG. 8 is thus an example of a class of embodiments in which each of at least part of the first conductor 301, at least part of the second conductor 302, and at least part of the third conductor 303 is embedded in a matrix material 350. The first sensing junction 316A, second sensing junction 316B, and second electrically conductive material 302 are furthermore flush with an outer surface 354 of the matrix material 350.

FIGS. 9 and 10 are graphs depicting data obtained using an apparatus 2 of the type depicted in FIG. 1 to measure the variation with time of a local temperature of a sample after application of a heating pulse via Joule heating applied through the second conductor 302. Four curves are shown in FIG. 9, respectively showing measured variation of the temperature of air, oil, amoxicillin and water. The four curves are highly distinct, even between the three liquid samples, demonstrating that this simple technique is capable of distinguishing with high sensitivity between different materials. FIG. 10 compares three samples having different concentrations of the antibiotic amoxicillin. Again, the three different concentrations lead to three clearly distinguishable thermal responses that can be used to distinguish between the different samples. FIG. 10 demonstrates how the approach can be used specifically to detect dilution of medicines such as antibiotics.

FIG. 11 depicts a further embodiment where the thermal response is measured at multiple points around a solid sample 6. In embodiments of this type a thermal response of the sample 6 to the heating is measured with the sensor element 2 in contact (e.g. in direct contact with a surface of the sample or in contact via a thin coupling layer or gel not forming an integral part of the sample 6) with a first region 411 on the surface of the sample 6 and a further thermal response of the sample 6 to the heating is measured using a further sensor element 402 in contact (e.g. in direct contact with a surface of the sample or in contact via a thin coupling layer or gel not forming an integral part of the sample 6) with a second region 412 on the surface of the sample 6. The second region 412 is separate (i.e. not in contact with or overlapping with) the first region 411. The further sensor element 402 may take any of the forms described above for the sensor element 2. Alternatively, the further sensor element 402 may have a simpler construction, being configured for example only to measure temperature (rather than being able to provide heating and measure temperature, as is possible in embodiments of the sensor element 2). The further sensor element 402 may thus comprise a thermocouple device of the type discussed above with reference to FIG. 3 for example. The provision of the further sensor element 402 makes it possible to measure a thermal response of the sample 6 to the heating provided by the sensor element 2 at a distance which is further away from the sensor element 2. The heat can thus be made to propagate through a large proportion of the sample 6 before reaching the further sensor element 402. This approach can enhance the ability of the method to sample properties of the sample 6 deep within the sample 6 and/or to measure thicker sample effectively. In an embodiment, the second region 412 is on an opposite side of the sample 6 to the first region 411, which provides a particularly efficient way of sampling properties of the sample 6 deep within the sample 6.

1. An apparatus for testing a sample, comprising:
a sensor element comprising a first conductor, a second conductor and a third conductor, wherein:
the first conductor, second conductor and third conductor are connected together electrically in series;
the first conductor is connected to the second conductor at a first sensing junction;
the second conductor is connected to the third conductor at a second sensing junction; and
the apparatus further comprises a measurement unit configured to:
apply heating to a sample under test via Joule heating in the second conductor, the Joule heating being generated by driving an electrical current in series through the first conductor, second conductor and third conductor;
measure a thermal response of the sample to the heating by measuring a potential difference between the first conductor and the third conductor, wherein the potential difference is influenced by a temperature gradient along the first conductor and a temperature gradient along the third conductor via the Seebeck effect; and
generate an indication of whether the sample satisfies a predetermined criterion based on the measured thermal response.

2. The apparatus of claim 1, wherein a majority of the electrical resistance in the circuit through which current is driven to provide the Joule heating is contributed by the second conductor.

3. The apparatus of claim 1, wherein the electrical resistance of the second conductor is at least 2 times higher than the electrical resistance of the first conductor and than the electrical resistance of the third conductor.

4. The apparatus of claim 1, wherein the second conductor is locally elongate and has a length that is at least 2 times longer than a shortest distance between the first sensing junction and the second sensing junction.

5. The apparatus of claim 4, wherein the second conductor comprises a spiral, a helix or a serpentine shape.

6. The apparatus of claim 1, wherein an average cross-sectional area of the second conductor is at least 2 times smaller than an average cross-sectional area of the first conductor and than an average cross-sectional area of the third conductor.

7. The apparatus of claim 1, wherein the first sensing junction is formed by welding of the first conductor to the second conductor and the second sensing junction is formed by welding of the second conductor to the third conductor.

8. The apparatus of claim 1, wherein the first conductor, second conductor and third conductor all have different compositions.

9. The apparatus of claim 8, wherein the first conductor and third conductor are respectively formed from one of the following pairs of materials:
chromel and constantan as for a type E thermocouple device;
iron and constantan as for a type J thermocouple device;
chromel and alumel as for a type K thermocouple device;
82% Ni/18% Mo and 99.2% Ni/0.8% Co, by weight, as for a type M thermocouple device;
Nicrosil and Nisil as for a type N thermocouple device;
copper and constantan as for a type T thermocouple;
70% Pt/30% Rh and 94% Pt/6% Rh, by weight, as for a type B thermocouple device;
87% Pt/13% Rh by weight and platinum as for a type R thermocouple device;
90% Pt/10% Rh by weight and platinum as for a type S thermocouple device;
95% W/5% Re and 74% W/26% Re, by weight, as for a type C thermocouple device;
97% W/3% Re and 75% W/25% Re, by weight, as for a type D thermocouple device; and
Tungsten and 74% W/26% Re by weight as for a type G thermocouple device.

10. The apparatus of claim 1, wherein:
each of at least part of the first conductor, at least part of the second conductor, and at least part of the third conductor is embedded in a matrix material; and
one or more of the following are flush with an outer surface of the matrix material and can be brought into direct contact with the sample: the first sensing junction, the second sensing junction, and the second conductor.

11. The apparatus of claim 1, wherein the heating of the sample and the measurement of the thermal response are performed in non-overlapping time periods.

12. A method of testing a sample, comprising:
providing a sensor element comprising a first conductor, a second conductor and a third conductor, wherein:
the first conductor, second conductor and third conductor are connected together electrically in series;
the first conductor is connected to the second conductor at a first sensing junction;
the second conductor is connected to the third conductor at a second sensing junction; and
the method further comprises:
applying heating to a sample under test via Joule heating in the second conductor, the Joule heating being generated by driving an electrical current in series through the first conductor, second conductor and third conductor;
measuring a thermal response of the sample to the heating by measuring a potential difference between the first conductor and the third conductor, wherein the potential difference is influenced by a temperature gradient along the first conductor and a temperature gradient along the third conductor via the Seebeck effect; and
generating an indication of whether the sample satisfies a predetermined criterion based on the measured thermal response.

13. The method claim 12, the heating of the sample and the measurement of the thermal response are performed in non-overlapping time periods.

14. The method of claim 12 wherein the sample comprises a pharmaceutical product.

15. The method of claim 12 wherein the sample comprises a liquid.

16. The method of 12, wherein the sample comprises a solid body.

17. The method of claim 16, wherein:
the thermal response of the sample to the heating is measured with the sensor element in contact with a first region on the sample; and
a further thermal response of the sample to the heating is measured using a further sensor element in contact with a second region on the sample, the second region being separate from the first region.

18. The method of claim 17, wherein the second region is on an opposite side of the sample to the first region.

19. The apparatus of claim 1, wherein the sample comprises a pharmaceutical product.

20. The apparatus of claim 1, wherein the sample comprises a liquid or a solid body.
